SEQUENCE LISTING (31 sequences)

SEQ ID NO: 1 - DNA, 403 bp, Cannabis sativa
  source 1..403: mol_type = other DNA
  variation 202: allele = n = g/a
aaattacagtttcacaaacattgggtacttatgccactaaaaataaacattgggtttatgccgcttacaaacataaaactttgggtacttatgccgctatttaccctaaaatttatatatttgtgcctgcctgtctttgacactagagcaaggttccatcatttctagcttggcagcaatgcgggctacacagccatcaacnacgttgttgagataaaccattggcgtattgccaatcaactacatgaaccaaaattaaaaaaagaaagccattagtaaagtagtatataactatatatagcaagcaaaagaagcatcataagattaagttatgacaaagataaattacttcagtagcgtctttcttgattgcgaacttgccctccatcttattctatttcaa

SEQ ID NO: 2 - DNA, 403 bp, Cannabis sativa
  source 1..403: mol_type = other DNA
  variation 202: allele = n = a/c
cctttgtcgacgtaagacattgacgatgaggaagtatagaatctgccagagagtgtatgcaaataagggaaccacaaagagccatgtccataggtaagatttgtcttctacataaggccatgaacttctgcgagaagatccttctggatgcatggctgcgaaggttgctggatcccaccatcgaatggtaaagaaaactatncctgtatcattttttatatacaagaaaaaataacattaaatagggtgtgataattggggatgccaagccaggataacaataaaaaacggatgactgaaccagatactacgctgctgctgctatacagcataaacgctatggctttgacaagccgataaaccaactagtagactttggtatggtgataaacattagagtcta

SEQ ID NO: 3 - DNA, 403 bp, Cannabis sativa
  source 1..403: mol_type = other DNA
  variation 202: allele = n = g/a
tgtgcttttttcagaagaaaataatagtaagagagaaattgtttggtctacaaactcgtctaaacaagccgcgaaaccattggttcagcttttggataatggtaacttggttttgagagacgagaaagatgcaaacacaacaaactatttatgggaaagctttgattatccaactgatacattgttgccaggaatgaaattnggatgggacttgaagagaggtctcaataggcgtttatcatcatggaagagcttttatagtgatccatgtgatggagatttcacttatgggattgagcttgatgagaaacaccatacatatcctcaactaactgtccgtaatggatctgcaatattgtatcgtcaagggttgtgggatggtatgagttccagcgatgctgtg

SEQ ID NO: 4 - DNA, 403 bp, Cannabis sativa
  source 1..403: mol_type = other DNA
  variation 202: allele = n = c/g
gggattttggaggaaattccttggtgggtttttggttttatgcgttttgggagtgggaggagaagtagaagaagaaccaccaacagcagcagcagcagcctcaccaccttgctccttcccggcgctgtacaactttggtgactcaaactccgataccggtggcatttcagctgcttttgaaccgattcctgcgccgtatggngaaggcttctttcataaaccatccggaagagactgtgatggccgtcttattgtagattttataggtaagatttaagtgaataagaactaactatataagattttgtgtattaacaacagagttggtgttgtttaattatgcagctgagcgcataggattaccatacttgagtgcttacttaaactcattaggaacaaatta

SEQ ID NO: 5 - DNA, 403 bp, Cannabis sativa
  source 1..403: mol_type = other DNA
  variation 202: allele = n = g/c
tcggctgggtttccgacagctgcgccgacgcctccggcgactaagccggctgcgatttttttggggagcgtgaaattgcccgtttggggatcggtccatttttgtttaaggacttcgtagagacccattcgggtggcggagtaaagggtttgacggaggattgtggcggagacgcctgagaagagagcggcgactccttcgntacggacgatgtttacccctatggatactgggccgacacgtggcggtggatccatgaggtggatggaatggacggcggagatgtttccgggcatggctggacggaataatgaagatgggtttggattaattggaggggtgagagtgggctcgccgtgaagctgcattcggaccttgataaggtcaagtgggtgggttgaag

SEQ ID NO: 6 - DNA, 403 bp, Cannabis sativa
  source 1..403: mol_type = other DNA
  variation 202: allele = n = a/g
atttattttgtatttaatacatttttttttaaaaaaaaaagacaagaaaaacagaaagatttagtatttgttacactaaagtatttattgatgaattgatttcacaaaagtgattaaattcttttccgatgagccaccttctctcatagcttctttagttaaattttgccattttttaacattttttacaatttctgtcctnttttcattttcgctcattactaactccaaacatctcttgatctcttcacttttaactattccatcttcatcgggctttactctaacacctattttccacatttcttctatcaacttagcattcgtctgttgatccgtccattgtggaaatgccaccactggaacccctgaaactatgctttctaacgtcgagttccaacca

SEQ ID NO: 7 - DNA, 403 bp, Cannabis sativa
  source 1..403: mol_type = other DNA
  variation 202: allele = n = t/c
tatgttcttttgggtgggatagtttaataaagttgtgttatgaagctagattgtttggtattgattaatctccttatcttgtcacacatactaatctttgttatgctgtcttttaacattatgtaaaagatcacaagtttatctttctcttcttcttggaatagcgtattccataatagacacattttagtgtaagttctcntgtttctcattatacttttgttcaatattttatagcagcataactctttacttgctcatctccatatgctgtcaccgtttaactgaaggaatacctgatctaacgatagtatccaattttagttgctgttcatcatccatcaagcattttgttgagtgtcaacttattgttcttaccgtgtatgttataagtatattctaa

SEQ ID NO: 8 - DNA, 20 bp, synthetic construct
  source 1..20: mol_type = other DNA
ttgtgcctgcctgtctttga

SEQ ID NO: 9 - DNA, 20 bp, synthetic construct
  source 1..20: mol_type = other DNA
atggagggcaagttcgcaat

SEQ ID NO: 10 - DNA, 20 bp, synthetic construct
  source 1..20: mol_type = other DNA
aagatggagggcaagttcgc

SEQ ID NO: 11 - DNA, 20 bp, synthetic construct
  source 1..20: mol_type = other DNA
tctgccagagagtgtatgca

SEQ ID NO: 12 - DNA, 20 bp, synthetic construct
  source 1..20: mol_type = other DNA
gcagcagcgtagtatctggt

SEQ ID NO: 13 - DNA, 21 bp, synthetic construct
  source 1..21: mol_type = other DNA
tctgccagagagtgtatgcaa

SEQ ID NO: 14 - DNA, 20 bp, synthetic construct
  source 1..20: mol_type = other DNA
tcgtctaaacaagccgcgaa

SEQ ID NO: 15 - DNA, 24 bp, synthetic construct
  source 1..24: mol_type = other DNA
tccattacggacagttagttgagg

SEQ ID NO: 16 - DNA, 23 bp, synthetic construct
  source 1..23: mol_type = other DNA
tggttcagcttttggataatggt

SEQ ID NO: 17 - DNA, 20 bp, synthetic construct
  source 1..20: mol_type = other DNA
catcccacaacccttgacga

SEQ ID NO: 18 - DNA, 20 bp, synthetic construct
  source 1..20: mol_type = other DNA
agaaccaccaacagcagcag

SEQ ID NO: 19 - DNA, 21 bp, synthetic construct
  source 1..21: mol_type = other DNA
tggtaatcctatgcgctcagc

SEQ ID NO: 20 - DNA, 20 bp, synthetic construct
  source 1..20: mol_type = other DNA
gaagaaccaccaacagcagc

SEQ ID NO: 21 - DNA, 20 bp, synthetic construct
  source 1..20: mol_type = other DNA
cattcgggtggcggagtaaa

SEQ ID NO: 22 - DNA, 20 bp, synthetic construct
  source 1..20: mol_type = other DNA
ttcaacccacccacttgacc

SEQ ID NO: 23 - DNA, 20 bp, synthetic construct
  source 1..20: mol_type = other DNA
attcgggtggcggagtaaag

SEQ ID NO: 24 - DNA, 20 bp, synthetic construct
  source 1..20: mol_type = other DNA
caacccacccacttgacctt

SEQ ID NO: 25 - DNA, 20 bp, synthetic construct
  source 1..20: mol_type = other DNA
tccgatgagccaccttctct

SEQ ID NO: 26 - DNA, 20 bp, synthetic construct
  source 1..20: mol_type = other DNA
gtttcaggggttccagtggt

SEQ ID NO: 27 - DNA, 20 bp, synthetic construct
  source 1..20: mol_type = other DNA
ccgatgagccaccttctctc

SEQ ID NO: 28 - DNA, 20 bp, synthetic construct
  source 1..20: mol_type = other DNA
tttcaggggttccagtggtg

SEQ ID NO: 29 - DNA, 22 bp, synthetic construct
  source 1..22: mol_type = other DNA
tgttcttttgggtgggatagtt

SEQ ID NO: 30 - DNA, 20 bp, synthetic construct
  source 1..20: mol_type = other DNA
acggtgacagcatatggaga

SEQ ID NO: 31 - DNA, 22 bp, synthetic construct
  source 1..22: mol_type = other DNA
atgttcttttgggtgggatagt

(19) United States
(12) Patent Application Publication
(10) Pub. No.: US 2026/0209869 A1
(43) Pub. Date: Jul. 23, 2026

(54) QUANTITATIVE TRAIT LOCI ASSOCIATED WITH FLOWER TO LEAF RATIO IN CANNABIS

(71) Applicant: PUREGENE AG, Zeiningen (CH)

(72) Inventors: Claudio Cropano, Zeiningen (CH); Dániel Árpád Carrera, Zeiningen (CH); Gavin Mager George, Zeiningen (CH); Leron Katsir, Zeiningen (CH); Maximilian Moritz Vogt, Zeiningen (CH); Michael Edward Ruckle, Zeiningen (CH); Michele Wyler, Zeiningen (CH)

(21) Appl. No.: 19/142,062

(22) PCT Filed: Dec. 22, 2023
(86) PCT No.: PCT/IB2023/063169

§ 371 Date: Jun. 20, 2025

(30) Foreign Application Priority Data
Dec. 22, 2022 (GB) 2219516.8

(51) Int. Cl. C12Q 1/6895; A01H 1/04

(52) U.S. Cl. CPC C12Q 1/6895; A01H 1/045; C12Q 2600/13; C12Q 2600/156

(57) ABSTRACT

The invention relates to methods of identifying and characterizing a Cannabis spp. plant with respect to a flower to leaf ratio trait comprising genotyping the plant for a quantitative trait locus (QTL) associated with a flower to leaf ratio trait, and to methods of producing plants having a flower to leaf ratio trait of interest based on defined allelic states of polymorphisms defining the QTL. Also provided are Cannabis spp. plants having a flower to leaf ratio trait of interest comprising defined allelic states of polymorphisms defining the QTL and plants identified, characterized or produced by the methods described herein. The invention further relates to marker assisted selection and marker assisted breeding methods, in particular using a combination of specific markers provided, for obtaining plants having a flower to leaf ratio trait of interest or for modulating the flower to leaf ratio of cannabis plants.

### BACKGROUND OF THE INVENTION

The invention relates to methods of identifying and characterizing a Cannabis spp. plant comprising a quantitative trait locus (QTL) associated with flower to leaf ratio, and to Cannabis spp. plants having a flower to leaf ratio trait of interest comprising defined allelic states of the polymorphisms defining the QTL. The invention further relates to plants with a flower to leaf ratio trait of interest identified by the methods described herein. The invention also relates to marker assisted selection and marker assisted breeding methods for obtaining plants having a flower to leaf ratio trait of interest. Also provided are methods of producing Cannabis spp. plants with the flower to leaf ratio trait of interest and plants produced by these methods, based on the allelic state of the QTLs.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are typically used for different purposes. Hemp is primarily used for industrial purposes, for example in feed, food, seed, fiber, and oil production. Conversely, HRT cannabis is largely cultivated and bred for high concentrations of the pharmacological constituents, cannabinoids, derived from resin in the trichomes.

Cannabis is the only species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to more than 30%.

Cannabis female flowers are highly enriched in cannabinoid content in comparison to other tissue types—this is likely mainly due to the abundance of trichomes on flowers enriched for cannabinoids. Because HRT cannabis is cultivated globally for flower production, identifying plants that produce increased flower yield can benefit cultivators.

The Cannabis Sativa L (cannabis) growing period comprises a vegetative phase followed by a reproductive, flower development phase. During long-day photoperiods, flowering is inhibited, and the plant remains in a vegetative growth phase. Seasonal changes will induce flowering when day length reduces to a minimum of 10-12 hours of continuous darkness.

Individual flowers occur as an apical inflorescence and at nodes along the stem and branches. Flowers appear as clusters, or compound inflorescences/raceme, as a result of higher order branching and shortened internodes and appear as compressed and dense while more distal inflorescences gradually decrease in size and density with a scattered appearance. In mature, compound inflorescences/racemes, also known colloquially as buds, nugs, or colas, these are tightly packed with stigmas curling over adjacent bracts at maturity.

Higher order branching also causes the leaflet number to gradually reduce from compound palmate “fan leaves” to smaller leaves usually with fewer leaflets emerging from the compound inflorescence. These reduced leaves emerging from the terminal inflorescences are colloquially known as sugar leaves because they are often coated with trichomes that appear “frosty”.

Varieties with high flower to leaf ratios produce buds that have many flowers and fewer leaves emerging. Although sugar leaves often contain significant numbers of trichomes they are almost always trimmed off during processing with the fan leaves. The genetic basis for high flower to leaf ratio is unknown in cannabis.

Understanding the genetic basis of flower to leaf ratio in cannabis can benefit the cannabis industry through the manipulation of this trait in a way that would benefit cultivators of hemp-type and HRT cannabis. Regulating this trait may result in HRT and Hemp type cannabis varieties with either high flower to leaf ratios, or low flower to leaf ratios. High flower to leaf ratios will benefit cultivators of medicinal and recreational cannabis as it will increase flower yield per plant and can simplify and expedite the trimming process thereby reducing the handling time and risk of contaminating flowers during processing and packaging. A high flower to leaf ratio is also attractive to consumers as it results in flowers that have a more homogeneous surface appearance and usually contain higher concentrations of cannabinoids. A low flower to leaf ratio can, however, also be beneficial to cultivators in countries like India where restrictive cannabis laws only allow the harvesting of cannabinoids from cannabis leaf tissue. In hemp-type cannabis leafy varieties may be desirable where cultivation is not for flower but for biomass in general.

In the present invention, several genetic regions in cannabis that significantly associate with cannabis flower to leaf ratio were identified from a parent population displaying segregation of the flower to leaf ratio trait and a method to identify the regions of the cannabis genome that contribute significantly to the trait was employed. The present invention thus relates to markers for the control of flower to leaf ratio in cannabis with the aim of developing varieties with a range of cannabis flower to leaf ratio phenotypes.

### SUMMARY OF THE INVENTION

The present invention describes methods of identifying and/or characterizing a Cannabis spp. plant with respect to a flower to leaf ratio trait comprising genotyping the plant for at least one quantitative trait locus (QTL) associated with a flower to leaf ratio trait, and to methods of producing plants having a flower to leaf ratio trait of interest based on defined allelic states of polymorphisms defining the QTL. Also described are Cannabis spp. plants having a flower to leaf ratio trait of interest comprising defined allelic states of polymorphisms defining the QTLs and plants identified, characterized, or produced by the methods described. The invention further relates to marker assisted selection and marker assisted breeding methods, in particular using a combination of markers provided herein, for obtaining plants having a flower to leaf ratio trait of interest or for modulating the flower to leaf ratio of cannabis plants.

According to a first aspect of the present invention there is provided a method for characterizing a Cannabis spp. plant with respect to a flower to leaf ratio trait, the method comprising the steps of: (i) genotyping at least one plant with respect to at least one flower to leaf ratio QTL by detecting one or more polymorphisms associated with the flower to leaf ratio trait as defined in Table 2; and (ii) characterizing the one or more plants with respect to the at least one flower to leaf ratio QTL as having an increased flower to leaf ratio QTL, a decreased flower to leaf ratio QTL or an intermediate flower to leaf ratio QTL, based on the genotype at the polymorphism.

In a first embodiment of the method for characterizing a Cannabis spp. plant with respect to a flower to leaf ratio trait, the polymorphism may be selected from the group consisting of “common_3494”, “common_567”, and combinations thereof, as defined in Table 2. These markers have all been validated for their predictive value for the flower to leaf ratio QTL and trait, particularly in combination.

In a second embodiment of the method for characterizing a Cannabis spp. plant with respect to a flower to leaf ratio trait, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms.

According to a third embodiment of the method for characterizing a Cannabis spp. plant with respect to a flower to leaf ratio trait, the molecular markers may be for detecting polymorphisms at regular intervals within the at least one flower to leaf ratio QTL such that recombination can be excluded. In an alternative embodiment, the molecular markers may be for detecting polymorphisms at regular intervals within the at least one flower to leaf ratio QTL such that recombination can be quantified to estimate linkage disequilibrium between a particular polymorphism and the flower to leaf ratio phenotype. It will be appreciated by those of skill in the art that several possible markers may be designed for detecting the polymorphisms. For example, molecular markers may be for detecting polymorphisms such that recombination events can be detected to a resolution of 10'000 or 100'000 or 500'000 base pairs within the QTL. In one embodiment, the molecular markers may be designed based on a context sequence for the polymorphism as provided in Table 4 herein, or the molecular markers may be selected from the primer pairs as defined in Table 5.

In a fourth embodiment of the method for characterizing a Cannabis spp. plant with respect to a flower to leaf ratio trait, the flower to leaf ratio QTL may be selected from one or more QTLs defined in Table 3 with reference to the CS10 reference genome and defined by one or more polymorphisms associated with the flower to leaf ratio trait as defined in Table 2, or a combination of such QTLs. In another embodiment, the flower to leaf ratio QTL may be defined by a genetic marker linked to one of the QTLs defined in Table 3.

According to a second aspect of the present invention, there is provided for a method of producing a Cannabis spp. plant having a flower to leaf ratio trait of interest, the method comprising the steps of: (i) providing a donor parent plant having in its genome at least one flower to leaf ratio QTL characterized by one or more polymorphisms associated with the flower to leaf ratio trait of interest as defined Table 2; (ii) crossing the donor parent plant having the at least one flower to leaf ratio QTL with at least one recipient parent plant to obtain a progeny population of cannabis plants; (iii) screening the progeny population of cannabis plants for the presence of the at least one flower to leaf ratio QTL; and (iv) selecting one or more progeny plants having the at least one flower to leaf ratio QTL, wherein the mature plant displays the flower to leaf ratio trait of interest. The flower to leaf ratio trait of interest may be an increased flower to leaf ratio trait, a decreased flower to leaf ratio trait, or an intermediate flower to leaf ratio trait. In this way, the trait can be modulated in a plant using the flower to leaf ratio QTLs and markers therefor described herein.

In a first embodiment of the method of producing a Cannabis spp. plant having a flower to leaf ratio trait of interest, the method may further comprise the steps of: (v) crossing the one or more progeny plants with the donor recipient plant; or (vi) selfing the one or more progeny plants.

According to a second embodiment of the method of producing a Cannabis spp. plant having a flower to leaf ratio trait of interest, the screening may comprise genotyping at least one plant from the progeny population with respect to the at least one flower to leaf ratio QTL by detecting one or more polymorphisms associated with the flower to leaf ratio trait of interest as defined Table 2.

In a third embodiment of the method of producing a Cannabis spp. plant having a flower to leaf ratio trait of interest, the method may comprise a step of genotyping the donor parent plant with respect to the at least one flower to leaf ratio QTL by detecting one or more polymorphisms associated with the flower to leaf ratio trait of interest as defined in Table 2, preferably prior to step (i).

According to a fourth embodiment of the method of producing a Cannabis spp. plant having a flower to leaf ratio trait of interest, the genotyping may be performed by a PCR-based detection method using molecular markers, by sequencing of PCR products containing the one or more polymorphisms, by targeted resequencing, by whole genome sequencing, or by restriction-based methods, for detecting the one or more polymorphisms.

In a fifth embodiment of the method of producing a Cannabis spp. plant having a flower to leaf ratio trait of interest, the molecular markers may be for detecting polymorphisms at regular intervals within the at least one flower to leaf ratio QTL such that recombination can be excluded. In an alternative embodiment, the molecular markers may be for detecting polymorphisms at regular intervals within the at least one flower to leaf ratio QTL such that recombination can be quantified to estimate linkage disequilibrium between a particular polymorphism and the flower to leaf ratio trait of interest. For example, molecular markers may be for detecting polymorphisms such that recombination events can be detected to a resolution of 10'000 or 100'000 or 500'000 base pairs within the QTL. It will be appreciated by those of skill in the art that several possible markers may be designed for detecting the polymorphisms. In one embodiment, the molecular markers may be designed based on a context sequence for the polymorphism described in Table 4 or may be selected from the primer pairs defined in Table 5.

According to a further embodiment of the method of producing a Cannabis spp. plant having a flower to leaf ratio trait of interest, the at least one flower to leaf ratio QTL is an increased flower to leaf ratio QTL, a decreased flower to leaf ratio QTL, or an intermediate flower to leaf ratio QTL defined by the allelic state of the polymorphisms as provided in Table 2. In one embodiment, the flower to leaf ratio trait of interest is an increased flower to leaf ratio trait, and the flower to leaf ratio QTL is an increased flower to leaf ratio QTL. Of particular use in producing a Cannabis spp. plant having a flower to leaf ratio trait of interest, are the polymorphisms selected from the group consisting of “common_3494”, “common_567”, and combinations thereof, as defined in Table 2, which have been validated for their predictive value for the flower to leaf ratio QTL and trait, and particularly to a combination of these polymorphisms.

According to a third aspect of the present invention there is provided for a method of producing a Cannabis spp. plant that has a flower to leaf ratio trait of interest, the method comprising introducing at least one flower to leaf ratio QTL characterized by one or more polymorphisms associated with the flower to leaf ratio trait of interest as defined in Table 2 into a Cannabis spp. plant, wherein said flower to leaf ratio QTL is associated with the flower to leaf ratio trait of interest in the plant. In one embodiment, introducing the at least one flower to leaf ratio QTL comprises crossing a donor parent plant having the at least one flower to leaf ratio QTL characterized by one or more polymorphisms associated with the flower to leaf ratio trait of interest with a recipient parent plant. In an alternative embodiment, introducing the at least one flower to leaf ratio QTL characterized by one or more polymorphisms associated with the flower to leaf ratio trait of interest comprises genetically modifying the Cannabis spp. plant. Several methods of genetic modification are known to those of skill in the art, including targeted mutagenesis, genome editing, and gene transfer. For example, a flower to leaf ratio QTL comprising one or more of the polymorphisms associated with the flower to leaf ratio trait of interest as defined in Table 2 herein may be introduced into a plant by mutagenesis and/or gene editing. In particular, the methods of genetically modifying a plant may be selected from the group consisting of CRISPR-Cas9 targeted gene editing, heterologous gene expression using various expression cassettes; TILLING, and non-targeted chemical mutagenesis using e.g., EMS. For example, CRISPR-Cas9 targeted gene editing may be achieved using a guide RNA. Alternatively, a cannabis spp. plant may be transformed with a cassette containing the flower to leaf ratio QTL associated with the flower to leaf ratio trait of interest or a part thereof, via any transformation method known in the art.

In one embodiment of the method of producing a Cannabis spp. plant that has a flower to leaf ratio trait of interest, the at least one flower to leaf ratio QTL is selected from one or more flower to leaf ratio QTLs defined in Table 3 with reference to the CS10 reference genome and defined by one or more polymorphisms associated with the flower to leaf ratio trait as defined in Table 2, or a combination thereof. In another embodiment, the flower to leaf ratio QTL may be defined by a genetic marker linked to the flower to leaf ratio QTL defined in Table 3.

According to a fourth aspect of the present invention there is provided for a Cannabis spp. plant characterized according to the method for characterizing a Cannabis spp. plant with respect to a flower to leaf ratio trait as described herein. In some embodiments, the Cannabis spp. plant characterized according to the method of a characterizing a Cannabis spp. plant having a flower to leaf ratio trait of interest as described herein is not exclusively obtained by means of an essentially biological process.

In a fifth aspect of the present invention there is provided for a Cannabis spp. plant produced according to the method of a producing a Cannabis spp. plant having a flower to leaf ratio trait of interest as described herein. In some embodiments, the Cannabis spp. plant produced according to the method of a producing a Cannabis spp. plant having a flower to leaf ratio trait of interest as described herein is not exclusively obtained by means of an essentially biological process.

According to a further aspect of the present invention there is provided for a Cannabis spp. plant comprising at least one flower to leaf ratio QTL characterized by one or more polymorphisms associated with a flower to leaf ratio trait of interest as defined in Table 2. In some embodiments, the plant is not exclusively obtained by means of an essentially biological process.

According to another aspect of the present invention there is provided for a quantitative trait locus that controls a flower to leaf ratio trait in Cannabis spp., wherein the quantitative trait locus is selected from one or more flower to leaf ratio QTLs defined in Table 3 with reference to the CS10 reference genome and defined by one or more polymorphisms associated with the flower to leaf ratio trait as defined in Table 2, or a genetic marker linked to the QTL. In some embodiments, the quantitative trait loci defined in Table 3 are provided as isolated nucleic acid molecule(s).

According to yet a further aspect of the present invention there is provided for a Cannabis spp. plant comprising one or more quantitative trait loci or a nucleic acid comprising said QTL as defined herein.

### BRIEF DESCRIPTION OF THE FIGURES

Non-limiting embodiments of the invention will now be described by way of example only and with reference to the following figures:

FIG. 1: Images of representative plants scored as having an increased flower to leaf ratio (score 7-9), intermediate flower to leaf ratio (score 4-6), and decreased flower to leaf ratio (score 1-3).

FIG. 2: Segregation of flower to leaf ratio in the F2 populations tested. The F2 population designation is shown above each plot. The Y-axis shows plant count, and the X-axis shows the mean flower to leaf ratio for each population-bars indicate standard deviation.

FIG. 3: A GWA of flower to leaf ratio from a combined Cannabis F2 population using the BLINK Model. Each box represents a separate chromosome, with the chromosome name above the plot and the relative position on the chromosome on the X-axis below, the Y-axis is the LOD score, −log₁₀(p).

FIG. 4: A Plot illustrating the selection of the model to fit the minimum number of markers used to predict the greatest variance in plant flower to leaf ratio. The X-axis represents the number of markers used for prediction. The Y-axis displays the model fit measured using the Bayesian information criterion (BIC).

### SEQUENCES

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

Methods are provided herein for identifying and obtaining plants having a flower to leaf ratio trait of interest, including an increased flower to leaf ratio trait, using a molecular marker detection technique. The inventors of the present invention have further produced and selected for flower to leaf ratio traits of interest in cannabis plants by crossing plants with various patterns of the flower to leaf ratio trait. Also demonstrated herein, the inventors were able to use genome wide association (GWA) to identify multiple QTLs linked to flower to leaf ratio and to show that these QTLs contribute in combination to flower to leaf ratio. The method provides evidence that the genetic basis of flower to leaf ratio is polygenic. This finding provides for the improvement of methods for producing plants displaying differing flower to leaf ratios.

A total of six (6) QTLs related to flower to leaf ratio were identified in the combined F2 populations tested.

Table 2 herein provides several single nucleotide polymorphisms (SNPs) which define the QTLs associated with a flower to leaf ratio trait of interest. In some embodiments one or more of the identified SNPs can be used to incorporate the increased flower to leaf ratio trait, decreased flower to leaf ratio trait, or intermediate flower to leaf ratio trait from a donor plant, containing one or more of the QTLs associated with the trait, into a recipient plant. For example, the incorporation of the flower to leaf ratio trait of interest may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising a series of polymorphisms in linkage disequilibrium are provided. The QTLs each display limited frequency of recombination within the QTLs. Preferably the polymorphisms are selected from Table 2 herein, representing the QTLs related to flower to leaf ratio variance. Molecular markers may be designed for use in detecting the presence of the polymorphisms and thus the QTLs. Further, the identified QTL polymorphisms and the associated molecular markers may be used in a cannabis breeding program to predict the flower to leaf ratio trait of interest of plants in a breeding population and can be used to produce cannabis plants that either display an increased flower to leaf ratio trait, decreased flower to leaf ratio trait, or intermediate flower to leaf ratio trait, compared to the plants from which they are derived.

As used herein, the term “flower to leaf ratio” refers to the number of flowers in comparison to the number of leaves emerging from the terminal inflorescence (cola) of a single plant at the time of harvest.

The “time of harvest” is defined with respect to the maturity of the flower, where approximately greater than 50% of the pistils have turned brown in appearance. The “time of harvest” can also be determined by initiation of flowering for hemp-type cannabis or by other agronomic criteria common in the art.

As used herein, reference to a plant or a variety with a “flower to leaf ratio trait” refers to a plant or a variety that shows a variation in flower to leaf ratio, conferred by the flower to leaf ratio QTLs identified herein.

As used herein, the term “flower to leaf ratio trait of interest” refers to the state of the plant with respect to the flower to leaf ratio trait at the time of harvest, and includes increased flower to leaf ratio, decreased flower to leaf ratio or intermediate flower to leaf ratio, compared with the plants from which they are derived.

As used herein, reference to a plant or variety with “increased flower to leaf ratio” or an “increased flower to leaf ratio trait” refers to a plant or variety that has a variation in flower to leaf ratio such that it has increased flower to leaf ratio, i.e., a higher number of flowers in comparison to the number of leaves, compared to the plants from which it is derived. In some cases, a plant or variety with increased flower to leaf ratio has a propensity for increased flower to leaf ratio in comparison to the mean flower to leaf ratio of plants from a population from which the plant or variety was derived.

As used herein, reference to a plant or variety with “decreased flower to leaf ratio” or a “decreased flower to leaf ratio trait” refers to a plant or variety that has a variation in flower to leaf ratio such that it has decreased flower to leaf ratio, i.e., fewer flowers in comparison to the number of leaves, compared to the plants from which it is derived. In some cases, a plant or variety with decreased flower to leaf ratio has a propensity for decreased flower to leaf ratio in comparison to the mean flower to leaf ratio of plants from a population from which the plant or variety was derived.

As used herein, reference to a plant or variety with “intermediate flower to leaf ratio” or an “intermediate flower to leaf ratio trait” refers to a plant or variety that has a variation in flower to leaf ratio such that it is has an intermediate flower to leaf ratio, i.e., about the average number of flowers in comparison to the number of leaves, when compared to the plants from which it is derived. In some cases, a plant or variety with intermediate flower to leaf ratio has a propensity for intermediate or a similar flower to leaf ratio in comparison to the mean flower to leaf ratio of plants from a population from which the plant or variety was derived.

It is a particular aim of the present invention to identify and characterize a plant for the flower to leaf ratio trait of interest early in the plant lifecycle, particularly prior to the plant displaying the flower to leaf ratio trait of interest, or to introduce a flower to leaf ratio trait of interest into a breeding population of plants early on in a breeding program. This can be achieved by genotyping the plants using molecular markers for detecting at least one QTL associated with the flower to leaf ratio trait of interest prior to the appearance of the trait.

As used herein a “quantitative trait locus” or “QTL” is a polymorphic genetic locus with at least two alleles that differentially affect the expression of a continuously varying phenotypic trait when present in a plant or organism which is characterised by a series of polymorphisms in linkage disequilibrium with each other.

As used herein, the term “flower to leaf ratio QTL” or “flower to leaf ratio quantitative trait locus” refers to a quantitative trait locus comprising part, or all, of the QTLs characterized by the polymorphisms having an allelic state associated with a flower to leaf ratio trait of interest, as described or defined in Table 2, or characterized by combinations of such polymorphisms. It is a particular aim of the present invention to provide plants with an increased flower to leaf ratio trait by genotyping and/or selecting the plants for an increased flower to leaf ratio QTL.

As used herein, the term “increased flower to leaf ratio QTL” or “increased flower to leaf ratio quantitative trait locus” refers to a quantitative trait locus characterized by one or more polymorphisms having an allelic state associated with increased flower to leaf ratio described or defined in Table 2.

In some cases, it is desirable to obtain a plant displaying an alternative flower to leaf ratio trait of interest, where it is desired to obtain a plant displaying a decreased flower to leaf ratio trait, or intermediate flower to leaf ratio trait. Increased flower to leaf ratio is particularly desirable for increased flower cannabinoid yield. However, an increased flower to leaf ratio may impart a growth penalty on plant development, which can include, among others, an impact on plant growth and yield of certain desirable cannabis products including fiber and seed. In particular, reducing plant flower to leaf ratio in some cannabis varieties may improve biomass yield and may also be beneficial for leaf cannabinoid production in countries where cannabinoids may only be harvested from leaves. Thus, depending on the application, the desirable flower to leaf ratio trait may be a decreased, intermediate, or increased flower to leaf ratio trait as described herein.

As used herein, the term “decreased flower to leaf ratio QTL” or “decreased flower to leaf ratio quantitative trait locus” refers to a quantitative trait locus characterized by one or more polymorphisms having an allelic state associated with decreased flower to leaf ratio as described or defined in Table 2.

As used herein, the term “intermediate flower to leaf ratio QTL” or “intermediate flower to leaf ratio quantitative trait locus” refers to a quantitative trait locus characterized by one or more polymorphisms having an allelic state associated with intermediate flower to leaf ratio described or defined in Table 2.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “flower to leaf ratio haplotype” refers to the subset of the polymorphisms contained within any one of the flower to leaf ratio QTLs which exist on a single haploid genome complement of the diploid genome, and which are in linkage disequilibrium with the flower to leaf ratio trait.

As used herein, the term “increased flower to leaf ratio haplotype” refers to the subset of the polymorphisms contained within any one of the increased flower to leaf ratio QTLs which exist on a single haploid genome complement of the diploid genome, and which are in linkage disequilibrium with the increased flower to leaf ratio trait.

As used herein, the term “donor parent plant” refers to a plant having a flower to leaf ratio haplotype, or one or more flower to leaf ratio alleles, associated with the flower to leaf ratio trait of interest.

As used herein, the term “recipient parent plant” refers to a plant having a flower to leaf ratio haplotype, or one or more flower to leaf ratio alleles, not associated with the flower to leaf ratio trait of interest.

The term “flower to leaf ratio allele” refers to the haplotype allele within a particular QTL that confers, or contributes to, the flower to leaf ratio trait of interest, or alternatively, is an allele that allows the identification of plants with the flower to leaf ratio trait of interest that can be included in a breeding program (“marker assisted breeding”, “marker assisted selection”, or “genomic selection”).

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the flower to leaf ratio trait.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the flower to leaf ratio trait. In the context of association studies, the genetic variants which are responsible for the association signal at a locus are referred to as the “causal variants”. Causal variants may comprise one or more “causal polymorphisms” that have a biological effect on the phenotype.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTLs of the invention, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid(s) identified herein, for example a nucleic acid carrying one or more of the QTLs defined herein, may be isolated nucleic acids, which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids, and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridization reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridization generally depends on the ability of denatured DNA to re-anneal when complementary strands are present in an environment below their melting temperature. A typical example of such “stringent” hybridization conditions would be hybridization carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for the Flower to Leaf Ratio Trait and Molecular Markers Therefor

In some embodiments, methods are provided for identifying a QTL or haplotype responsible for a flower to leaf ratio trait of interest, such as the increased, decreased or intermediate flower to leaf ratio trait, thereby to identify the QTL or haplotype responsible for the trait, and for selecting plants with the flower to leaf ratio trait of interest. In some embodiments, the methods may comprise the steps of:
a. Identifying a plant that displays a flower to leaf ration trait of interest, such as an increased flower to leaf ratio trait, a decreased flower to leaf ratio trait or an intermediate flower to leaf ratio trait, within a breeding program.
b. Establishing a population by crossing the identified plant to itself (selfing) or a recipient parent plant.
c. Genotyping the resultant F1, or subsequent populations, for example by sequencing methods.
d. Performing association studies, including phenotyping and linkage analysis, to discover QTLs and/or polymorphisms contained within the QTL.
e. Optionally, identifying cannabis paralogs of previously characterized genes that may be involved in conferring the flower to leaf ratio trait of interest.
f. Developing molecular markers that detect one or more polymorphisms linked to QTLs, alleles within these QTLs, or existing or induced polymorphisms.
g. Validating the molecular markers by determining the linkage disequilibrium between the marker and the increased, decreased, or intermediate flower to leaf ratio trait.

### Trait Development and Introgression

In some embodiments, methods are provided for marker assisted breeding (MAB) or marker assisted selection (MAS) of plants having a flower to leaf ratio QTL or displaying the flower to leaf ratio trait of interest. The methods may comprise the steps of:
a Identifying a plant that displays the flower to leaf ratio trait of interest or phenotype or contains a flower to leaf ratio QTL associated with the flower to leaf ratio trait of interest as defined herein.
b. Establishing a population by crossing the identified plant to itself (selfing) or another recipient parent plant.
C. Genotyping and phenotyping the resultant F1, or subsequent, populations, for example by sequencing methods.
d. Performing association studies, inputting phenotype and genotype information to identify genomic regions enriched with polymorphisms associated with the flower to leaf ratio trait of interest, to discover QTLs and/or polymorphisms contained within the QTL.
e Optionally, identifying cannabis paralogs of previously characterized genes that may be involved in conferring the flower to leaf ratio trait of interest.
f. Developing molecular markers that detect one or more polymorphisms linked to QTLs, alleles within these QTLs, or existing or induced polymorphisms.
g. Using the molecular markers when introgressing the QTLs or polymorphisms into new or existing cannabis varieties to select plants containing the flower to leaf ratio of interest, or a flower to leaf ratio haplotype associated with the flower to leaf ratio trait of interest.

### QTLs and Marker Assisted Breeding

In some embodiments, during the breeding process, selection of plants displaying the flower to leaf ratio of interest as described herein may be based on molecular markers designed to detect polymorphisms linked to genomic regions that control the trait of interest by either an identified or an unidentified mechanism. Previously identified genetic mechanisms may, for example, have a direct or pleiotropic effect on the flower to leaf ratio of a plant. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the flower to leaf ratio trait of interest, based on identification of polymorphisms that are either linked to, or found within QTLs that are associated with the flower to leaf ratio trait of interest using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the phenotype of the flower to leaf ratio trait of interest are identified within synthetic populations of plants capable of revealing dominant, recessive, or complex traits. In one embodiment of the invention, a genetically diverse population of cannabis varieties, that are used to produce the synthetic population are integrated into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the following methods are employed to integrate the plants into a breeding program including some or all of the following:
a. Growing plants in rich media or soils under artificial lighting;
b. Cloning of plants, often through a multitude of sub-cloning cycles;
C. Introduction of plants into in vitro, sterile growth environments, and subsequent removal to standard growth conditions;
d. Exposure to mutagens such as EMS, colchicine, silver nitrate, ethidium bromide, dinitroanilines, high concentrations of mono- or poly-chromatic light sources;
e. Growing plants under highly stressful conditions which include restricted space, drought, pathogen challenge, atypical temperatures, and nutrient stresses.

### Flower to Leaf Ratio Trait of Interest Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a flower to leaf ratio trait of interest may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest, and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the increased flower to leaf ratio phenotype, decreased flower to leaf ratio phenotype or intermediate flower to leaf ratio phenotype of the plants. Using the association studies described herein, together with accurate phenotyping, this method is able to identify genomic regions, QTLs and even specific genes or polymorphisms responsible for the flower to leaf ratio trait of interest that are directly introduced into recipient lines. Polygenic phenotypes may also be identified using the methods described herein.

In one embodiment, the structured population is grown to the time of harvest. To characterize the phenotypes of the lines, they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can be used to improve the accuracy of genomic selection models. In some embodiments, a genomic selection model that incorporates flower to leaf ratio can be improved by the inclusion of the flower to leaf ratio QTLs in the GS model. In some embodiments, the SNPs described in Table 2 may be useful in a genomic selection model, for example where genotypes with unknown phenotypes are evaluated using an approach like a random forest algorithm for prediction of the flower to leaf ratio trait, and particularly in combination, to improve the predictive power of the model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system, including primers designed based on the context sequences for the polymorphisms identified herein, provided in Table 4. Such molecular markers may include PCR primers, or targeted sequencing primers such as those described in the examples below, more specifically the primers defined in Table 5.

For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the QTL and by association, the flower to leaf ratio trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the flower to leaf ratio trait of interest.

According to some embodiments, any polymorphism in linkage disequilibrium with one or more of the flower to leaf ratio QTLs can be used to determine the flower to leaf ratio haplotype in a breeding population of plants, as long as the polymorphism is unique to the flower to leaf ratio trait of interest in the donor parent plant when compared to the recipient parent plant.

In some embodiments, the desired trait may be the increased flower to leaf ratio trait, and the donor parent plant may be a plant that has been genetically modified or selected to include an increased flower to leaf ratio QTL defined by a polymorphism conferring the increased flower to leaf ratio trait, for example any, some, or all of the polymorphisms defined in Table 2 associated with the trait.

Alternatively, the desired trait may be the intermediate- or decreased flower to leaf ratio trait, and the donor parent plant may be a plant that has been genetically modified or selected to include a an intermediate- or decreased flower to leaf ratio QTL defined by a polymorphism associated with intermediate- or decreased flower to leaf ratio, for example any, some, or all of the polymorphisms defined in Table 2 associated with the trait.

In some embodiments, donor parent plants, as described above, are used as one of two parents to create breeding populations (F1) through sexual reproduction. In this embodiment, donor parent plants may be identified by detecting polymorphisms using the molecular markers as described above.

Methods for reproduction that are known in the art may be used. The donor parent plant provides the trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the flower to leaf ratio allele or flower to leaf ratio haplotype associated with the flower to leaf ratio trait of interest in plants to be used in the F1 cross is determined using the described molecular markers. In some embodiments, the resulting F2 progeny, or subsequent progeny, is/are screened for any of the polymorphisms associated with the flower to leaf ratio trait of interest, and in particular polymorphisms associated with the increased flower to leaf ratio trait of interest, described herein.

The plants at any generation can be produced by asexual means like cutting and cloning, or any method that yields a genetically identical offspring.

Production of Cannabis Spp. Plants Having the Increased Flower to Leaf Ratio Trait

In some embodiments, a Cannabis spp. plant that has the decreased flower to leaf ratio trait or intermediate flower to leaf ratio trait may be converted into a plant having an increased flower to leaf ratio trait according to the methods of the present invention by providing a breeding population where the donor parent plant contains an increased flower to leaf ratio QTL associated with an increased flower to leaf ratio and the recipient parent plant either displays the decreased- or intermediate flower to leaf ratio trait or contains the decreased- or intermediate flower to leaf ratio QTL.

In some embodiments the decreased flower to leaf ratio trait or intermediate flower to leaf ratio trait may be removed from a recipient parent plant by crossing it with a donor parent plant having the increased flower to leaf ratio QTL. In some embodiments the donor parent plant has an increased flower to leaf ratio phenotype and contains a contiguous genomic sequence characterized by one or more of the polymorphisms of Table 2 associated with the increased flower to leaf ratio allele or haplotype.

In some embodiments, the donor parent plant is any cannabis variety that is cross fertile with the recipient parent plant.

In some embodiments, MAS or MAB may be used in a method of backcrossing plants carrying the increased flower to leaf ratio trait to a recipient parent plant. For example, an F1 plant from a breeding population can be crossed again to the recipient parent plant. In some embodiments, this method is repeated.

In some embodiments, the resulting plant population is then screened for the increased flower to leaf ratio trait using MAS with molecular markers to identify progeny plants that contain one or more polymorphism, such as any of those described in Table 2, indicating the presence of an allele of a QTL associated with the increased flower to leaf ratio phenotype. In another embodiment, the population of cannabis plants may be screened by any analytical methods known in the art to identify plants with desired characteristics, specifically the increased flower to leaf ratio trait.

Production of Cannabis Spp. Plants Having an Intermediate- or Decreased Flower to Leaf Ratio Trait

In some embodiments, a Cannabis spp. plant that has the increased flower to leaf ratio trait may be converted into a plant having an intermediate- or decreased flower to leaf ratio trait according to the methods of the present invention by providing a breeding population where the donor parent plant contains a decreased- or intermediate flower to leaf ratio QTL and recipient parent plant either displays the increased flower to leaf ratio phenotype or contains the increased flower to leaf ratio QTL.

In some embodiments the increased flower to leaf ratio trait may be removed from a recipient parent plant by crossing it with a donor parent plant having the decreased- or intermediate flower to leaf ratio QTL. In some embodiments the donor parent plant has a decreased- or intermediate flower to leaf ratio phenotype and contains a contiguous genomic sequence characterized by one or more of the polymorphisms of Table 2 associated with the decreased flower to leaf ratio allele or haplotype, or the intermediate flower to leaf ratio allele or haplotype.

In some embodiments, the donor parent plant is any cannabis variety that is cross fertile with the recipient parent plant.

In some embodiments, MAS or MAB may be used in a method of backcrossing plants carrying the decreased- or intermediate flower to leaf ratio trait to a recipient parent plant. For example, an F1 plant from a breeding population can be crossed again to the recipient parent plant. In some embodiments, this method is repeated.

In some embodiments, the resulting plant population is then screened for the intermediate- or decreased flower to leaf ratio trait using MAS with molecular markers to identify progeny plants that contain one or more polymorphism, such as any of those described Table 2, indicating the presence of an allele of a QTL associated with the intermediate- or decreased flower to leaf ratio phenotype. In another embodiment, the population of cannabis plants may be screened by any analytical methods known in the art to identify plants with desired characteristics, specifically the decreased- or intermediate flower to leaf ratio trait.

### Methods to Genetically Engineer Plants to Achieve the Flower to Leaf Ratio Trait of Interest Using Mutagenesis or Gene Editing Techniques

Identifying QTLs, and individual polymorphisms, that correlate with a trait when measured in an F1, F2, or similar breeding population indicates the presence of one or more causative polymorphisms in close proximity to the polymorphism detected by the molecular marker. In some embodiments, the polymorphisms associated with the flower to leaf ratio trait of interest are introduced into a plant by other means so that a trait, such as the increased-, decreased- or intermediate flower to leaf ratio trait, can be introduced into plants that would not otherwise contain associated causative polymorphisms or removed from plants that would otherwise contain associated causative polymorphisms. For example, the polymorphisms detailed in Table 2 are molecular markers that can be used to indicate the presence of a possible causative polymorphism.

The entire QTLs or parts thereof which confer the flower to leaf ratio trait of interest described herein may be introduced into the genome of a cannabis plant to obtain plants with increased flower to leaf ratio, decreased flower to leaf ratio or intermediate flower to leaf ratio, through a process of genetic modification known in the art, for example, but not limited to, heterologous gene expression using an expression cassette including a sequence encoding the QTL(s) or part thereof, or the nucleic acids comprising them. The expression cassette may contain all or part of the QTL(s), including possible causative polymorphisms.

The flower to leaf ratio trait of interest described herein may be removed from, or introduced into, the genome of a cannabis spp. plant to obtain plants that exclude or include the causative polymorphisms and the potential to display a desired flower to leaf ratio trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the flower to leaf ratio trait of interest described in Table 2 or a causative polymorphism linked thereto. Non limiting examples of SSNs include zinc finger nucleases (ZFNs), TAL effector nucleases (TALENs), meganuclease, and, clustered regularly interspaced short palindromic repeats (CRISPR)/CRISPR-associated protein (Cas) system. In some embodiments, non-limiting examples of Cas proteins suitable for use in the methods of the present invention include Csn1, Cpf1 Cas9, Cas 12, Cas 13, Cas 14, CasX and combinations thereof. In one embodiment, a modified Cannabis spp. plant with the desired flower to leaf ratio is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the flower to leaf ratio trait of interest, such as a polymorphism associated with the flower to leaf ratio trait of interest described in Table 2, or a causative polymorphism linked thereto.

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The gRNA may be designed based on the context sequence for the polymorphisms provided in Table 4. The predefined gene specific gRNAs may be cloned into the same plasmid as the Cas gene and this plasmid is inserted into plant cells as described above.

In some embodiments, once the guide RNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion the gene coding sequence for a gene responsible for the flower to leaf ratio trait of interest is disrupted and the translation of the encoded protein is compromised either by a premature stop codon or disruption of a functional or structural property of the protein.

In another embodiment, the flower to leaf ratio trait of interest in Cannabis spp. plants may be introduced by generating gRNA with homology to a specific site of predetermined genes in the Cannabis genome or the QTLs defined herein. This gRNA may be sub-cloned into a plasmid containing the Cas9 gene, and the plasmid inserted into the Cannabis plant cells. In this way site specific mutations in the QTLs are generated, particularly causative polymorphisms linked to a SNP associated with the flower to leaf ratio trait of interest described in Table 2, thus effectively modulating, including increasing or decreasing, the flower to leaf ratio in the genome edited plant.

In some embodiments, a modified Cannabis spp. plant exhibiting the flower to leaf ratio trait of interest may be obtained using the targeted genome modification methods described above, wherein the plant comprises a targeted genome modification to introduce one or more polymorphisms associated with the flower to leaf ratio trait of interest, particularly a causative polymorphism linked to a SNP defined in Table 2, wherein the modification effects a flower to leaf ratio trait of interest.

In some embodiments, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with molecular markers as described herein to identify transgenic individuals with an increased-, intermediate-, or decreased flower to leaf ratio QTL or polymorphism(s), following the genetic modification.

In some embodiments, cannabis spp. plants having one or more of the polymorphisms of Table 2 associated with increased-, intermediate-, or decreased flower to leaf ratio QTLs or a causative polymorphism linked thereto are provided. The polymorphisms may be introduced, for example, by genetic engineering. In some embodiments the one or more polymorphisms associated with the flower to leaf ratio trait of interest, or a causative polymorphism linked thereto are introduced into the plants by breeding, such as by MAS or MAB, for example as described herein.

The nucleic acid molecules comprising the increased-, intermediate-, or decreased flower to leaf ratio QTLs herein responsible for conferring an increased-, intermediate-, or decreased flower to leaf ratio trait, may be under the control of, or operably linked to, a promoter, for example an inducible promoter. Such nucleic acid molecules may be operably linked to the inducible promoter so as to induce or suppress the flower to leaf ratio trait or phenotype in the plant or plant cell.

Accordingly, in a further embodiment, Cannabis spp. plants comprising an increased-, intermediate-, or decreased flower to leaf ratio QTL described herein, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Flower to Leaf Ratio in Cannabis

To identify the genetic basis of flower to leaf ratio in cannabis, diverse population of cannabis displaying variation in flower to leaf ratio, including in hemp-types and resin-types, were assembled and grown in a field trial in a 2020 in Niederwil, Switzerland. Genotypes that displayed variation in flower to leaf ratio were used as a basis to generate a number of F2 populations. During an outdoor field trial in 2021 select members of these F2 populations, predicted to show segregation for flower to leaf ratio, were grown to maturity and scored for flower to leaf ratio. To find regions of the genome that associate with variation in flower to leaf ratio the inventors conducted a genome wide association study using scores derived from the evaluation of cannabis flower to leaf ratio at the time of harvest in comparison to a reference scoreboard.

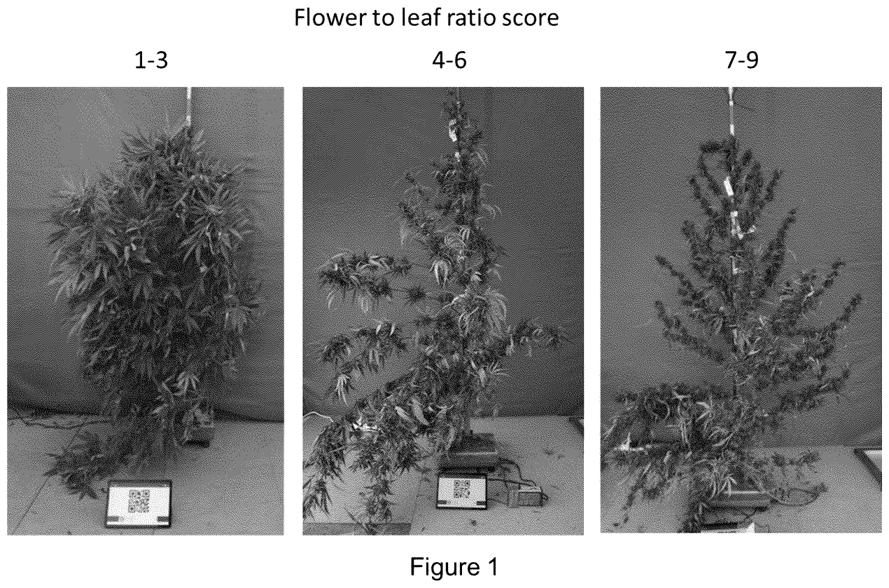

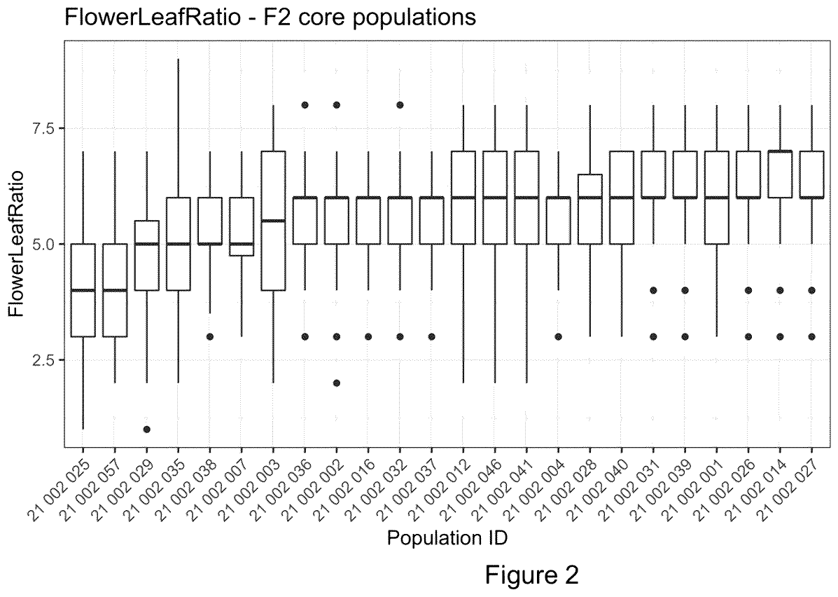

The inventors sought to understand the genetic basis for flower to leaf ratio by recording the flower to leaf ratio scores of 24 designed F2 populations, comprising in total 2434 individuals. At the end of the 2021 field season that started on Aug. 3, 2021, an image of each full cannabis plant was captured with a Canon EOS M6 II using a Canon EF-M 11-22 mm f/4-5.6 IS STM lens. A scoreboard was created with plant images representative of high flower to leaf ratio (score 7-9), intermediate flower to leaf ratio (score 4-6), and low flower to leaf ratio (score 1-3) (FIG. 1). Each plant was evaluated for flower to leaf ratio based on the score board (FIG. 2). The recorded data for flower to leaf ratio was filtered for duplicates and outliers. After cleaning, summary statistics data count, mean, and standard deviation was computed for each F2 population (Table 1). The mean flower to leaf ratio score for the 24 F2 populations ranged from 6.8-3.7, with a maximum flower to leaf ratio number recorded for individual plants of 9 and a minimum of 1, indicating a large degree of phenotypic variation found in the F2 populations created. An overview of the flower to leaf ratio distribution is given for the 24 selected F2 populations in FIG. 2 and in Table 1.

TABLE 1
A representative overview of the F2 populations after filtering used in determining
flower to leaf ratio. including the population identifier (ID). the average flower to leaf ratio
based on the score (Mean). the standard deviation of the mean flower to leaf ratio (StDev).
the minimum (Min). maximum (Max) and median flower to leaf ratio based on the scoring for
that population as well as the sample size of the population given by the number of plants
(Number).

| ID | Mean | StDev | Min | Max | Median | Number |
|---|---|---|---|---|---|---|
| 21 002 001 | 6.14 | 1.06 | 3 | 8 | 6 | 152 |
| 21 002 002 | 5.47 | 1.14 | 2 | 8 | 6 | 129 |
| 21 002 003 | 5.39 | 1.43 | 2 | 8 | 5.5 | 110 |
| 21 002 004 | 5.79 | 0.94 | 3 | 7 | 6 | 111 |
| 21 002 007 | 5.38 | 1.14 | 3 | 7 | 5 | 76 |
| 21 002 012 | 5.67 | 1.39 | 2 | 8 | 6 | 137 |
| 21 002 014 | 6.24 | 1.24 | 3 | 8 | 7 | 76 |
| 21 002 016 | 5.61 | 0.99 | 3 | 7 | 6 | 83 |
| 21 002 025 | 3.75 | 1.42 | 1 | 7 | 4 | 81 |
| 21 002 026 | 6.18 | 1.07 | 3 | 8 | 6 | 90 |
| 21 002 027 | 6.29 | 1.09 | 3 | 8 | 6 | 85 |
| 21 002 028 | 5.79 | 1.05 | 3 | 8 | 6 | 107 |
| 21 002 029 | 4.62 | 1.31 | 1 | 7 | 5 | 99 |
| 21 002 031 | 6.07 | 0.98 | 3 | 8 | 6 | 76 |
| 21 002 032 | 5.62 | 1.05 | 3 | 8 | 6 | 88 |
| 21 002 035 | 5.2 | 1.47 | 2 | 9 | 5 | 142 |
| 21 002 036 | 5.43 | 1.25 | 3 | 8 | 6 | 113 |
| 21 002 037 | 5.66 | 0.95 | 3 | 7 | 6 | 80 |
| 21 002 038 | 5.28 | 0.99 | 3 | 7 | 5 | 106 |
| 21 002 039 | 6.08 | 0.93 | 3 | 8 | 6 | 107 |
| 21 002 040 | 6 | 1.04 | 3 | 7 | 6 | 93 |
| 21 002 041 | 5.74 | 1.21 | 2 | 8 | 6 | 90 |
| 21 002 046 | 5.68 | 1.21 | 2 | 8 | 6 | 113 |
| 21 002 057 | 3.94 | 1.12 | 2 | 7 | 4 | 90 |

Following scoring for the flower to leaf ratio, DNA from a subset of the 24 F2 populations described in part in Table 1 were sequenced. DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific. The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. Primers for the SNPs identified can be designed from the context sequences provided in Table 4, or the primers provided in Table 5. The library pool was loaded onto Ion 550 chips with Ion Chef and sequenced with Ion GeneStudio S5 Plus according to the manufacturer's instructions (Ion 550™ Kit from Thermo Fisher Scientific).

Using the 24 combined F2 populations with a total of 2434 individuals, a genome-wide association study (GWAS) was performed to detect significant associations between genotypic information derived from targeted resequencing of the custom SNP marker panel described above with flower to leaf ratio score. The recorded data on flower to leaf ratio for the F2 populations were used as an input for GWAS.

For better modelling the inventors reasoned that a high rate of missing values may be impacting the estimation of population structure and kinship among individuals. To solve this, an additional step was incorporated. The GWA with all F2 populations combined was performed again but using a SNP matrix that underwent a round of imputation for reducing the number of missing values. In order to reduce missing data in the genotype file, an imputation was performed using the HapMap_imputation software (GitHub-mwylerCH/HapMap_Imputation). Briefly, the genotype file is converted to a hapmap format (comma separated, http://augustogarcia.me/statgen-esalq/Hapmap-and-VCF-formats-and-its-integration-with-onemap/#hapmap).

In a first step, HapMap_Imputation counts the occurrence of each nucleotide at every single genotyped position. The most common nucleotide is defined as major allele, the second is defined as minor allele. Missing genotyping information is excluded. In the case where major and minor alleles occur at the same number, the nucleotide of the cs10 reference genome is chosen as the major allele. Subsequently, HapMap_Imputation sorts markers by position and parses the hapmap into the required fastPHASE (Scheet and Stephens, 2006) input format. Briefly, HapMap_Imputation splits the haplotypes into two separate rows, converts major and minor alleles into 0 and 1 respectively and produces temporary files for each chromosome.

During the third step, HapMap_Imputation downloads the latest fastPHASE version and runs the imputation using 8 cores in parallel. fastPHASE is run with ten random starts of the imputation algorithm. After imputation, HapMap_Imputation reverses the 0 and 1 coding into the major and minor nucleotide, respectively. Subsequently, the two haplotypes are combined, and the separate chromosomes are merged into a single file.

The genotypic matrix was filtered for SNPs having more than 30% missing values within the population and a minor allele frequency lower than 5%. This resulted in 5077 SNP markers after filtering. The GWAS was performed using GAPIT version 3 (Wang and Zhang, 2021) with four statistical models: General Linear Model (GLM), Mixed Linear Model (MLM), FarmCPU and Blink. A quantile-quantile plot (QQ plot) was used to evaluate the statistical models. The Blink model performed the best by the inventors' evaluation and was used for the analysis. SNPs surpassing a LOD (−log₁₀(p-value)) value of 5 were considered to have a significant association with trait variation.

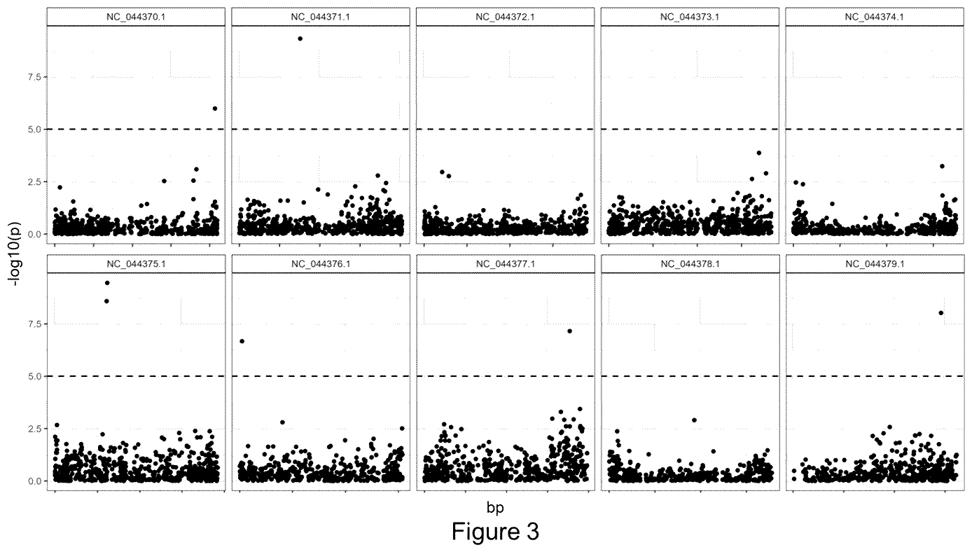

Flower to leaf ratio is a trait that is composed of many quantitative traits, and it was not clear that using flower to leaf ratio as a score to conduct a GWA would result in identifying associated SNPs. Unexpectedly, the inventors were able to identify 7 SNPs showing a significant association with variance in flower to leaf ratio, with an LOD value greater than 5 in the BLINK model in the imputed GWA, the positions are given with reference to the Cannabis sativa CS10 genome (FIG. 3 and Table 2). Based on the results of the imputed GWA the inventors identified 6 flower to leaf ratio QTLS, FLQTL1-6 (Table 3). The results of the GWA support the polygenic nature of flower to leaf ratio in cannabis. The polygenic nature of this trait underlies why breeding for flower to leaf ratio can be challenging.

Several SNPs (“common_567”, “common_5467”, and “GBScompat_rare_98”) stood out because the variation in flower to leaf ratio associated with different allele states at these SNPs, greater than 0.5, was by far larger than at the other SNPs found associated with flower to leaf ratio. These specific markers with the greatest variance represent excellent markers for designing plants with an increased- or decreased flower to leaf ratio trait. The largest variance was found for the SNP “common_567” where the AA variant had a mean flower to leaf ratio of 5.69 while the alternative GG variant had a mean flower to leaf ratio of 3.52. This suggests that this SNP underlies a QTL that significantly changes the flower to leaf ratio. The inventors found that the SNP most significantly associated with variance in flower to leaf ratio determined by the highest LOD score in the BLINK model is “GBScompat_rare_98”.

The homozygous allele of the SNPs in Table 2 that can be used to predict the propensity for increased or decreased flower to leaf ratio in cannabis plants are listed along with their position on the CS10 reference genome. Included also are the possible alleles at this position and the corresponding average flower to leaf ratio. The reference or context sequence for each of the SNPs identified is provided in Table 4 with reference to the CS10 genome.

In Table 5, PCR primers designed to amplify each of the regions containing these SNPs, with reference to the CS10 genome, are provided in order for the allelic variant to be determined.

TABLE 2
SNPs associated with flower to leaf ratio in the F2 populations. Mean increased flower
to leaf ratio is predicted by the occurrence of the indicative allele (marked with *). The positions
and chromosome of the SNPs are provided with reference to the CS10 reference genome as
described herein. The LOD score is provided as LOD. “Mean_1”, “Mean_2” and “Mean_3”
denote the average phenotypic value associated with Allele_1, Allele_2 and Allele_3,
respectively. based on scoring for flower to leaf ratio. Count_1, Count_2, and Count_3 denote
the number of plants that contributed to the average phenotypic value of Mean_1, Mean_2, and
Mean_3, respectively.

| SNP | Chromosome | Position | LOD | Alleles_1 | Alleles_2 | Alleles_3 |
|---|---|---|---|---|---|---|
| GBScompat_rare_98 | NC_044375.1 | 30775325 | 9.46087275 | AA | AG* | GG |
| GBScompat_common_150 | NC_044371.1 | 37815488 | 9.32582498 | AA | AC* | CC |
| common_3056 | NC_044375.1 | 30380938 | 8.58674119 | AA | AG | GG* |
| common_5467 | NC_044379.1 | 58426853 | 8.02574947 | CC | CG | GG* |
| common_4464 | NC_044377.1 | 70824628 | 7.15800952 | CC* | CG | GG |
| common_3494 | NC_044376.1 | 889215 | 6.67335678 | AA* | AG | GG |
| common_567 | NC_044370.1 | 103443430 | 5.99160751 | AA* | AG | GG |
| SNP | Mean_1 | Mean_2 | Mean_3 | Count_1 | Count_2 | Count_3 |
| GBScompat_rare_98 | 5.62440191 | 5.74419954 | 5.08027079 | 836 | 862 | 517 |
| GBScompat_common_150 | 5.50724638 | 5.68697124 | 5.48677686 | 414 | 591 | 1210 |
| common_3056 | 5.48840666 | 5.71662469 | 5.72794118 | 1682 | 397 | 136 |
| common_5467 | 5.16556291 | 5.60116086 | 5.83722287 | 755 | 603 | 857 |
| common_4464 | 5.62013348 | 5.13694268 | 5.45631068 | 1798 | 314 | 103 |
| common_3494 | 5.69106566 | 5.46826347 | 5.38137472 | 929 | 835 | 451 |
| common_567 | 5.65914546 | 4.01851852 | 3.52564103 | 2083 | 54 | 78 |

TABLE 3
Flower to leaf ratio QTLs (FLQTLs) associated with flower
to leaf ratio are given based on the SNPs identified
in Table 2 and the chromosome and position are provided
with reference to the CS10 reference genome.

| QTL | SNP | Chromosome |
|---|---|---|
| FLQTL1 | common_567 | NC_044370.1 |
| FLQTL2 | GBScompat_common_150 | NC_044371.1 |
| FLQTL3 | common_3056, GBScompat_rare_98 | NC_044375.1 |
| FLQTL4 | common_3494 | NC_044376.1 |
| FLQTL5 | common_4464 | NC_044377.1 |
| FLQTL6 | common_5467 | NC_044379.1 |

TABLE 4
Detailed information of each of the SNPs associated with flower to leaf ratio in
Cannabis as provided in Table 2. The “Ref” reference allele based on the CS10 genome, and
the identified “Alt” alternative allele based on the SNP marker panel are given for each SNP.
The “context sequence” is provided with the SNP given in square brackets. All of the sequences
and alleles are provided with reference to the plus strand.

| SNP | Ref | Alt | Context sequence |
|---|---|---|---|
| GBScompat_ | G | A | AAATTACAGTTTCACAAACATTGGGTACTTATGCCACTAAAAATAAACATTG |
| rare_98 | | | GGTTTATGCCGCTTACAAACATAAAACTTTGGGTACTTATGCCGCTATTTAC |
| | | | CCTAAAATTTATATATTTGTGCCTGCCTGTCTTTGACACTAGAGCAAGGTTC |
| | | | CATCATTTCTAGCTTGGCAGCAATGCGGGCTACACAGCCATCAAC[G/A]AC |
| | | | GTTGTTGAGATAAACCATTGGCGTATTGCCAATCAACTACATGAACCAAAA |
| | | | TTAAAAAAAGAAAGCCATTAGTAAAGTAGTATATAACTATATATAGCAAGCA |
| | | | AAAGAAGCATCATAAGATTAAGTTATGACAAAGATAAATTACTTCAGTAGCG |
| | | | TCTTTCTTGATTGCGAACTTGCCCTCCATCTTATTCTATTTCAA |
| | | | (SEQ ID NO: 1) |
| GBScompat_ | A | C | CCTTTGTCGACGTAAGACATTGACGATGAGGAAGTATAGAATCTGCCAGA |
| common_150 | | | GAGTGTATGCAAATAAGGGAACCACAAAGAGCCATGTCCATAGGTAAGAT |
| | | | TTGTCTTCTACATAAGGCCATGAACTTCTGCGAGAAGATCCTTCTGGATGC |
| | | | ATGGCTGCGAAGGTTGCTGGATCCCACCATCGAATGGTAAAGAAAACTAT |
| | | | [A/C]CCTGTATCATTTTTTATATACAAGAAAAAATAACATTAAATAGGGTGTG |
| | | | ATAATTGGGGATGCCAAGCCAGGATAACAATAAAAAACGGATGACTGAAC |
| | | | CAGATACTACGCTGCTGCTGCTATACAGCATAAACGCTATGGCTTTGACAA |
| | | | GCCGATAAACCAACTAGTAGACTTTGGTATGGTGATAAACATTAGAGTCTA |
| | | | (SEQ ID NO: 2) |
| common_3056 | G | A | TGTGCTTTTTTCAGAAGAAAATAATAGTAAGAGAGAAATTGTTTGGTCTACA |
| | | | AACTCGTCTAAACAAGCCGCGAAACCATTGGTTCAGCTTTTGGATAATGGT |
| | | | AACTTGGTTTTGAGAGACGAGAAAGATGCAAACACAACAAACTATTTATGG |
| | | | GAAAGCTTTGATTATCCAACTGATACATTGTTGCCAGGAATGAAATT[G/A]G |
| | | | GATGGGACTTGAAGAGAGGTCTCAATAGGCGTTTATCATCATGGAAGAGC |
| | | | TTTTATAGTGATCCATGTGATGGAGATTTCACTTATGGGATTGAGCTTGAT |
| | | | GAGAAACACCATACATATCCTCAACTAACTGTCCGTAATGGATCTGCAATA |
| | | | TTGTATCGTCAAGGGTTGTGGGATGGTATGAGTTCCAGCGATGCTGTG |
| | | | (SEQ ID NO: 3) |
| common_5467 | C | G | GGGATTTTGGAGGAAATTCCTTGGTGGGTTTTTGGTTTTATGCGTTTTGGG |
| | | | AGTGGGAGGAGAAGTAGAAGAAGAACCACCAACAGCAGCAGCAGCAGCC |
| | | | TCACCACCTTGCTCCTTCCCGGCGCTGTACAACTTTGGTGACTCAAACTCC |
| | | | GATACCGGTGGCATTTCAGCTGCTTTTGAACCGATTCCTGCGCCGTATGG |
| | | | [C/G]GAAGGCTTCTTTCATAAACCATCCGGAAGAGACTGTGATGGCCGTCTT |
| | | | ATTGTAGATTTTATAGGTAAGATTTAAGTGAATAAGAACTAACTATATAAGA |
| | | | TTTTGTGTATTAACAACAGAGTTGGTGTTGTTTAATTATGCAGCTGAGCGC |
| | | | ATAGGATTACCATACTTGAGTGCTTACTTAAACTCATTAGGAACAAATTA |
| | | | (SEQ ID NO: 4) |
| common_4464 | G | C | TCGGCTGGGTTTCCGACAGCTGCGCCGACGCCTCCGGCGACTAAGCCGG |
| | | | CTGCGATTTTTTTGGGGAGCGTGAAATTGCCCGTTTGGGGATCGGTCCAT |
| | | | TTTTGTTTAAGGACTTCGTAGAGACCCATTCGGGTGGCGGAGTAAAGGGT |
| | | | TTGACGGAGGATTGTGGCGGAGACGCCTGAGAAGAGAGCGGCGACTCCT |
| | | | TCG[G/C]TACGGACGATGTTTACCCCTATGGATACTGGGCCGACACGTGG |
| | | | CGGTGGATCCATGAGGTGGATGGAATGGACGGCGGAGATGTTTCCGGGC |
| | | | ATGGCTGGACGGAATAATGAAGATGGGTTTGGATTAATTGGAGGGGTGAG |
| | | | AGTGGGCTCGCCGTGAAGCTGCATTCGGACCTTGATAAGGTCAAGTGGGT |
| | | | GGGTTGAAG |
| | | | (SEQ ID NO: 5) |
| common_3494 | A | G | ATTTATTTTGTATTTAATACATTTTTTTTTAAAAAAAAAAGACAAGAAAAACA |
| | | | GAAAGATTTAGTATTTGTTACACTAAAGTATTTATTGATGAATTGATTTCACA |
| | | | AAAGTGATTAAATTCTTTTCCGATGAGCCACCTTCTCTCATAGCTTCTTTAG |
| | | | TTAAATTTTGCCATTTTTTAACATTTTTTACAATTTCTGTCCT[A/G]TTTTCATT |
| | | | TTCGCTCATTACTAACTCCAAACATCTCTTGATCTCTTCACTTTTAACTATTC |
| | | | CATCTTCATCGGGCTTTACTCTAACACCTATTTTCCACATTTCTTCTATCAA |
| | | | CTTAGCATTCGTCTGTTGATCCGTCCATTGTGGAAATGCCACCACTGGAAC |
| | | | CCCTGAAACTATGCTTTCTAACGTCGAGTTCCAACCA |
| | | | (SEQ ID NO: 6) |
| common_567 | T | C | TATGTTCTTTTGGGTGGGATAGTTTAATAAAGTTGTGTTATGAAGCTAGATT |
| | | | GTTTGGTATTGATTAATCTCCTTATCTTGTCACACATACTAATCTTTGTTATG |
| | | | CTGTCTTTTAACATTATGTAAAAGATCACAAGTTTATCTTTCTCTTCTTCTTG |
| | | | GAATAGCGTATTCCATAATAGACACATTTTAGTGTAAGTTCTC[T/C]TGTTTC |
| | | | TCATTATACTTTTGTTCAATATTTTATAGCAGCATAACTCTTTACTTGCTCAT |
| | | | CTCCATATGCTGTCACCGTTTAACTGAAGGAATACCTGATCTAACGATAGT |
| | | | ATCCAATTTTAGTTGCTGTTCATCATCCATCAAGCATTTTGTTGAGTGTCAA |
| | | | CTTATTGTTCTTACCGTGTATGTTATAAGTATATTCTAA |
| | | | (SEQ ID NO: 7) |

TABLE 5
Targeted sequencing primers (5′ to 3′) for the SNPs identified
in Table 2, as described in Example 1.

| SNP | Forward primer 1 | Reverse primer 1 | Forward primer 2 | Reverse primer 2 |
|---|---|---|---|---|
| GBScompat_ | TTGTGCCTGCCT | ATGGAGGGCAA | TTGTGCCTGCCT | AAGATGGAGGG |
| rare_98 | GTCTTTGA | GTTCGCAAT | GTCTTTGA | CAAGTTCGC |
| | (SEQ ID NO: 8) | (SEQ ID NO: 9) | (SEQ ID NO: 8) | (SEQ ID NO: 10) |
| GBScompat_ | TCTGCCAGAGA | GCAGCAGCGTA | TCTGCCAGAGA | GCAGCAGCGTA |
| common_150 | GTGTATGCA | GTATCTGGT | GTGTATGCAA | GTATCTGGT |
| | (SEQ ID NO: 11) | (SEQ ID NO: 12) | (SEQ ID NO: 13) | (SEQ ID NO: 12) |
| common_3056 | TCGTCTAAACAA | TCCATTACGGAC | TGGTTCAGCTTT | CATCCCACAACC |
| | GCCGCGAA | AGTTAGTTGAGG | TGGATAATGGT | CTTGACGA |
| | (SEQ ID NO: 14) | (SEQ ID NO: 15) | (SEQ ID NO: 16) | (SEQ ID NO: 17) |
| common_5467 | AGAACCACCAA | TGGTAATCCTAT | GAAGAACCACC | TGGTAATCCTAT |
| | CAGCAGCAG | GCGCTCAGC | AACAGCAGC | GCGCTCAGC |
| | (SEQ ID NO: 18) | (SEQ ID NO: 19) | (SEQ ID NO: 20) | (SEQ ID NO: 19) |
| common_4464 | CATTCGGGTGG | TTCAACCCACCC | ATTCGGGTGGC | CAACCCACCCA |
| | CGGAGTAAA | ACTTGACC | GGAGTAAAG | CTTGACCTT |
| | (SEQ ID NO: 21) | (SEQ ID NO: 22) | (SEQ ID NO: 23) | (SEQ ID NO: 24) |
| common_3494 | TCCGATGAGCC | GTTTCAGGGGTT | CCGATGAGCCA | TTTCAGGGGTTC |
| | ACCTTCTCT | CCAGTGGT | CCTTCTCTC | CAGTGGTG |
| | (SEQ ID NO: 25) | (SEQ ID NO: 26) | (SEQ ID NO: 27) | (SEQ ID NO: 28) |
| common_567 | TGTTCTTTTGGG | ACGGTGACAGC | ATGTTCTTTTGG | ACGGTGACAGC |
| | TGGGATAGTT | ATATGGAGA | GTGGGATAGT | ATATGGAGA |
| | (SEQ ID NO: 29) | (SEQ ID NO: 30) | (SEQ ID NO: 31) | (SEQ ID NO: 30) |

### Example 2

### Marker Selection for Flower to Leaf Ratio

Quantitative traits like flower to leaf ratio are highly complex and can be regulated by multiple genes or QTLs distributed across the genome. This poses a challenge in identifying specific markers that contribute most significantly to the trait of interest. In the case of flower to leaf ratio, 6 QTLs were identified (Table 3). The QTLs together with the polymorphism information provided in Table 2 can be used to identify plants with an allelic variant associated with a flower to leaf ratio of interest, providing an industrially applicable tool for selecting plants with these traits preferentially, for improving genomic selection models, and as part of a molecular marker breeding strategy. However, such approaches are not always amenable to such large numbers of QTLs. The inventors took an additional approach to identify the minimum number of QTLs, or SNPs, that contribute the most to the phenotypic variation of flower to leaf ratio using a combination of two different methods.

First, using the values for variation in flower to leaf ratio a regression analysis was conducted to first model and then predict the phenotype using the allelic status of all significant markers identified by the GWAS. The regression analysis conducted is based on the random forest algorithm (Breiman, 2001) as implemented in the ranger package (v.0.12.1 Wright and Ziegler, 2017) using the allele status as a factor. The subset was defined using the markers with the highest variable importance in the model.

To complement the above-described machine learning approach, the inventors deployed the exhaustive Leaps and Bounds variable selection (Furnival and Wilson, 2000) implemented in the leaps package (v.3.1, Lumley, 2020). Shortly, Leaps and Bounds performs a targeted variable sampling and calculates the model predictability with an increasing number of predictors. The described markers were identified as the main contributors to the model predictability.

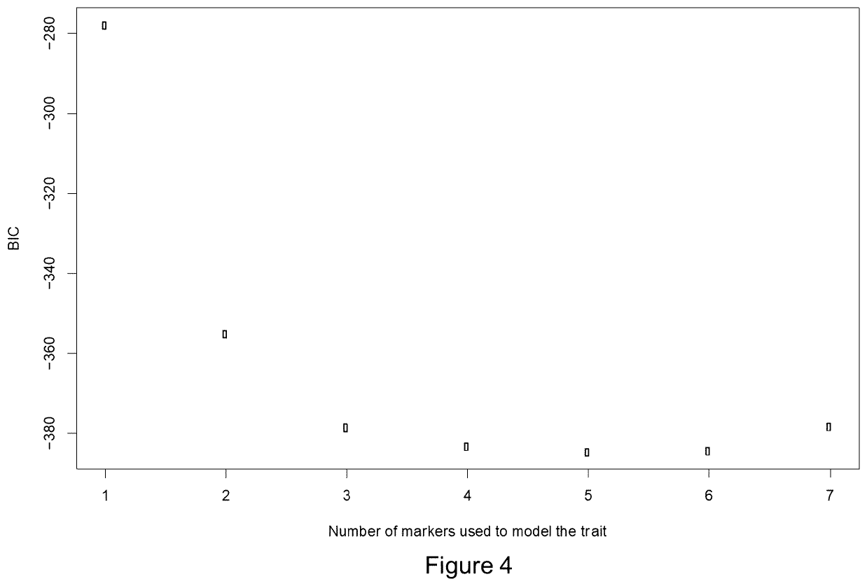

Based on the modeling the inventors found a best fit model that accounted for the most variation with the minimum number of markers. The model with two markers composed of “common_3494” and “common_567” accounted for most of the phenotypic variation found in flower to leaf ratio in the F2 populations used in the present invention (FIG. 4). The allelic variation associated with flower to leaf ratio of these two SNPs can be used to identify plants with an average propensity for both increased and decreased flower to leaf ratio, allowing the for selection of plants that produce more flowers where an increased flower to leaf ratio is desirable.

In the mixed F2 population plants, using the allelic combinations predicted to show the flower to leaf ratio per plant, the inventors were able to identify plants with the allelic variants of each of the two best fit markers that on average predicted for the largest variation in flower to leaf ratio. The inventors identified plants (Table 6) that contain the allelic variants: “common_3494”=AA and “common_567”=AA where the mean flower to leaf ratio would be predicted to be increased the most with respect to the average based on the predicted variation described in Table 2 and the results above. The 782 plants with these allelic variants were found to have a mean flower to density of 5.89 indicating a high flower to leaf ratio. This demonstrates that by selecting plants with these alleles, the mean flower to leaf ratio can be selectively increased on average. Combining this approach with selecting for additional alleles in Table 2 can lead to better selections for increased flower to leaf ratio. Alternatively, when either of the two SNPs alone, or in combination, were in the alternative allele states: “common_3494”=GG and “common_567”=GG plants with these variants were found to have much lower flower to leaf ratio (Table 2 and 6). For example, the 12 plants found with the allelic variants “common_3494”=GG and “common_567”=GG had a flower to leaf ratio of 3.1, indicating a low flower to leaf ratio. Combining this approach with selecting for additional alleles in Table 2 can lead to better selections for decreased flower to leaf ratio.

The allelic states and mean flower to leaf ratio for the two SNPs that account for most of the variation in flower to leaf ratio as described is given in Table 6.

TABLE 6
Combinatorial effect of SNPs on flower to leaf ratio variation.
Allelic variants of “common_3494” and “common_{—}
567” were evaluated for their effect in combination on
average flower to leaf ratio. Mean flower to leaf ratio indicates
the flower to leaf ratio composed of a given number of plants
(n) where that allele state combination for the SNPs is found.

| common_3494 | common_567 | Mean flower to leaf ratio | n |
|---|---|---|---|
| GG | GG | 3.0875 | 12 |
| AG | GG | 3.61692308 | 13 |
| GG | AG | 3.4 | 5 |
| AA | GG | 4.00217391 | 23 |
| AA | AG | 4 | 32 |
| AG | AG | 4.2 | 5 |
| GG | AA | 5.47106227 | 273 |
| AG | AA | 5.47973592 | 568 |
| AA | AA | 5.83920716 | 782 |

### REFERENCES

Breiman, L., “Random Forests,” Machine Learning, 45, (2001): 5-32.
Furnival, G. M. and Wilson, R .W., “Regressions by leaps and bounds,” Technometrics 42. (2000): 69-79. 10.2307/1271435.
Lumley, T. and Miller, A. J., “leaps: Regression Subset Selection.” (2004).
Scheet, P. and Stephens, M., “A fast and flexible statistical model for large-scale population genotype data: applications to inferring missing genotypes and haplotypic phase.” Am J Hum Genet. 2006 April; 78(4):629-44. doi: 10.1086/502802.
Wang, J and Zhang, Z., “GAPIT Version 3: Boosting Power and Accuracy for Genomic Association and Prediction,” Genomics, Proteomics & Bioinformatics 19, Issue 4 (2021): 629-640, https://doi.org/10.1016/j.gpb.2021.08.005.
Wright, M. N. and Ziegler, A., “ranger: A Fast Implementation of Random Forests for High Dimensional Data in C++ and R,” Journal of Statistical Software 77, no. 1 (2017): 1-17.

1. A method for characterizing a Cannabis spp. plant with respect to a flower to leaf ratio trait, the method comprising the steps of:
(i) genotyping at least one plant with respect to at least one flower to leaf ratio QTL by detecting one or more polymorphisms associated with the flower to leaf ratio trait as defined in Table 2; and
(ii) characterizing the one or more plants with respect to the at least one flower to leaf ratio QTL as having an increased flower to leaf ratio QTL, a decreased flower to leaf ratio QTL or an intermediate flower to leaf ratio QTL based on the genotype at the polymorphism.

2. The method of claim 1, wherein the polymorphism is selected from the group consisting of “common_3494”, “common_567”, and combinations thereof, as defined in Table 2.

3. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

4. The method of claim 3, wherein the molecular markers are for detecting polymorphisms at regular intervals within the at least one flower to leaf ratio QTL such that recombination can be excluded, or wherein the molecular markers are for detecting polymorphisms at regular intervals within the at least one flower to leaf ratio QTL such that recombination can be quantified to estimate linkage disequilibrium between a particular polymorphism and the flower to leaf ratio phenotype, optionally wherein the molecular markers are designed based on a context sequence for the polymorphism in Table 4 or are selected from the primer pairs as defined in Table 5.

5. (canceled)

6. (canceled)

7. The method of claim 1, wherein the at least one flower to leaf ratio QTL is selected from one or more QTLs defined in Table 3 with reference to the CS10 reference genome and is defined by one or more polymorphisms associated with the flower to leaf ratio trait as defined in Table 2, or a genetic marker linked to the QTL.

8. A method of producing a Cannabis spp. plant having a flower to leaf ratio trait of interest, the method comprising the steps of:
(i) providing a donor parent plant having in its genome at least one flower to leaf ratio QTL characterized by one or more polymorphisms associated with the flower to leaf ratio trait of interest as defined in Table 2;
(ii) crossing the donor parent plant having the at least one flower to leaf ratio QTL with at least one recipient parent plant to obtain a progeny population of cannabis plants;
(iii) screening the progeny population of cannabis plants for the presence of the at least one flower to leaf ratio QTL; and
(iv) selecting one or more progeny plants having the at least one flower to leaf ratio QTL, wherein the mature plant displays the flower to leaf ratio trait of interest.

9. The method of claim 8, further comprising:
(v) crossing the one or more progeny plants with the donor recipient plant; or
(vi) selfing the one or more progeny plants.

10. The method of claim 8, wherein the screening comprises genotyping at least one plant from the progeny population with respect to the at least one flower to leaf ratio QTL by detecting one or more polymorphisms associated with the flower to leaf ratio trait of interest as defined in Table 2, and optionally wherein the method further comprises a step of genotyping the donor parent plant with respect to the at least one flower to leaf ratio QTL by detecting one or more polymorphisms associated with the flower to leaf ratio trait of interest as defined in Table 2, prior to step (i).

11. (canceled)

12. The method of claim 10, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

13. The method of claim 12, wherein the molecular markers are for detecting polymorphisms at regular intervals within the at least one flower to leaf ratio QTL such that recombination can be excluded or such that recombination can be quantified to estimate linkage disequilibrium between a particular polymorphism and the flower to leaf ratio trait of interest, optionally wherein the molecular markers are designed based on a context sequence for the polymorphism in Table 4 or are selected from the primer pairs as defined in Table 5.

14. (canceled)

15. The method of claim 8, wherein the at least one flower to leaf ratio QTL is an increased flower to leaf ratio QTL, a decreased flower to leaf ratio QTL, or an intermediate flower to leaf ratio QTL.

16. The method of claim 8, wherein the polymorphism is selected from the group consisting of “common_3494”, “common_567”, and combinations thereof, as defined in Table 2.

17. The method of claim 8, wherein the branch number QTL is selected from one or more QTLs defined in Table 3 with reference to the CS10 reference genome and is defined by one or more polymorphisms associated with the flower to leaf ratio trait as defined in Table 2, or a genetic marker linked to the QTL.

18. A method of producing a Cannabis spp. plant that has a flower to leaf ratio trait of interest, the method comprising introducing at least one flower to leaf ratio QTL characterized by one or more polymorphisms associated with the flower to leaf ratio trait of interest as defined in Table 2 into a Cannabis spp. plant, wherein said QTL is associated with the flower to leaf ratio trait of interest in the plant.

19. The method of claim 18, wherein introducing the at least one flower to leaf ratio QTL comprises crossing a donor parent plant having the at least one flower to leaf ratio QTL characterized by one or more polymorphisms associated with the flower to leaf ratio trait of interest with a recipient parent plant.

20. The method of claim 18, wherein introducing the at least one flower to leaf ratio QTL characterized by one or more polymorphisms associated with flower to leaf ratio trait of interest comprises genetically modifying the Cannabis spp. plant.

21. The method of claim 18, wherein the at least one flower to leaf ratio QTL is selected from one or more QTLs defined in Table 3 with reference to the CS10 reference genome and is defined by one or more polymorphisms associated with the flower to leaf ratio trait as defined in Table 2, or a genetic marker linked to the QTL.

22. (canceled)

23. (canceled)

24. (canceled)

25. A Cannabis spp. plant comprising at least one flower to leaf ratio QTL characterized by one or more polymorphisms associated with a flower to leaf ratio trait of interest as defined in Table 2, wherein said flower to leaf ratio QTL is associated with the flower to leaf ratio trait of interest in the plant.

26. (canceled)

27. (canceled)
